(19) United States
(12) Patent Application Publication
(10) Pub. No.: US 2012/0153201 A1
(43) Pub. Date: Jun. 21, 2012

(54) LUER ACCESS VALVE

(75) Inventors: JACQUES ANDRE LAROSE, Round Lake, IL (US); Peter Karas, Barrington, IL (US)

(73) Assignee: Amsino International, Inc., Pomona, CA (US)

(21) Appl. No.: 12/974,153

(22) Filed: Dec. 21, 2010

(51) Int. Cl. F16K 31/44

(52) U.S. Cl. 251/213

(57) ABSTRACT

A valve system that comprises a housing having an opening for receiving or ejecting a fluid, a poppet that controls a flow of the fluid through the opening, an elastoguide that drives the poppet to a closed position, and an adaptor that receives and holds the poppet and the elastoguide.

### FIELD OF THE DISCLOSURE

This disclosure is directed to a male luer access valve that may be used with intravenous delivery devices, or the like.

### BACKGROUND OF THE DISCLOSURE

It is common practice to deliver various solids and/or fluids intravenously to patients. Intravenous delivery may include, for example, placing a cannula directly into the patient's vein or artery to deliver the fluids and medications. A similar mechanism may be used to withdraw fluid from the patient's vein or artery.

Where prolonged or repeated delivery (or withdrawal) of fluids and/or solids is anticipated, a vascular access site may be established in the patient. The vascular access site may include, for example, a sealed entry port that is attached directly to a needle or catheter. The sealed entry port may include a latex septum or latex plug, which is configured to receive a needle to access the patient's system with little pain or discomfort to the patient.

Luer-activated valves, including valves that use positive displacement technology, are being used with ever-increasing frequency. These valves tend to reduce the occurrence of complications such as, for example, catheter occlusions that may occur when using needles or cannulas. However, these luer-activated valves may be susceptible to cracking, misalignment, binding, and the like.

The present disclosure provides a luer access valve that is resistant to cracking or binding, requires less material, is simple in design and operation, and is easier and less expensive to manufacture.

### SUMMARY OF THE DISCLOSURE

According to an aspect of the disclosure, a valve system is disclosed that comprises: a housing that includes an opening for receiving or ejecting a fluid; a poppet that is configured to control a flow of the fluid through the opening; an elastoguide that is configured to drive the poppet to a closed position; and an adaptor that is configured to receive and hold the poppet and the elastoguide. The housing may comprise: a longitudinal housing portion and an offset housing portion, the longitudinal housing portion comprising an angle of between about 10° and about 15°; a fastener portion that is configured to engage and lock to a male luer; a plurality of channels that are configured to guide the fluid from one end of the housing to an opposite end of the housing; and/or a plurality of ribs that are configured to guide and hold the adaptor in the housing and to provide a positive stop for the poppet in the closed position. The poppet may comprise: a substantially hour-glass shape; an angled face at a housing-side end portion; and/or a surface that is configured to engage a portion of the elastoguide. The male luer may comprise a slip and lock style luer. The elastoguide may comprise an elastic material. The elastoguide may comprise: a recess that is configured reduce overall weight of the elastoguide or to provide additional compressibility; and/or a substantially longitudinal shape. The adaptor may comprise: a cavity that is configured to receive and hold a portion of the poppet; and/or a flange that engages a portion of the housing. The cavity may be further configured to receive and hold the elastoguide. The adaptor may comprise an opening that is configured to receive the fluid. The opening may be in fluid communication with a channel in the housing and a channel that is provided in the adaptor.

According to a further aspect of the disclosure, a valve system is disclosed, comprising: a housing that includes an opening for receiving or ejecting a fluid; a poppet that is configured to control a flow of the fluid through the opening; and an elastoguide that is configured to drive the poppet to a closed position, wherein the elastoguide includes a recess that is configured to reduce overall weight of the elastoguide or to provide additional compressibility. The valve system may further comprise an adaptor that is configured to hold the elastoguide.

According to a still further aspect of the disclosure, a method is disclosed for assembling a valve system. The method comprises: inserting an elastoguide in an adaptor; inserting the poppet in the adaptor with the elastoguide; and inserting the adaptor with the poppet and the elastoguide into a housing.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows a perspective view of an example of a valve system that is constructed according to the principles of the disclosure;

FIG. 2 shows a perspective view of another example of a valve system that is constructed according to the principles of the disclosure;

FIG. 3 shows an exploded view of the valve system of FIG. 1;

FIG. 4 shows an exploded view of the valve system of FIG. 2;

FIG. 5 shows a perspective view of an example of a housing of the valve system of FIG. 1;

FIGS. 6A-6B show a side view and a top view, respectively, of the housing of FIG. 5;

FIGS. 6C-6G show various views of another example of a housing that is constructed according to the principles of the disclosure;

FIGS. 7A-7C show various views of an example of a housing of the valve system of FIG. 2;

FIGS. 7D-7F show various views of a another example of a housing of the valve system of FIG. 2;

FIG. 8 shows an example of a poppet, which is constructed according to the principles of the disclosure;

FIG. 9 shows an example of an elastoguide, which is constructed according to the principles of the disclosure;

FIGS. 10A-10 show various views of an example of an adaptor, which is constructed according to the principles of the disclosure; and

FIG. 11 shows a process for assembling the valve system of FIG. 1 or FIG. 2.

The present disclosure is further described in the detailed description that follows.

### DETAILED DESCRIPTION OF THE DISCLOSURE

The embodiments of the disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

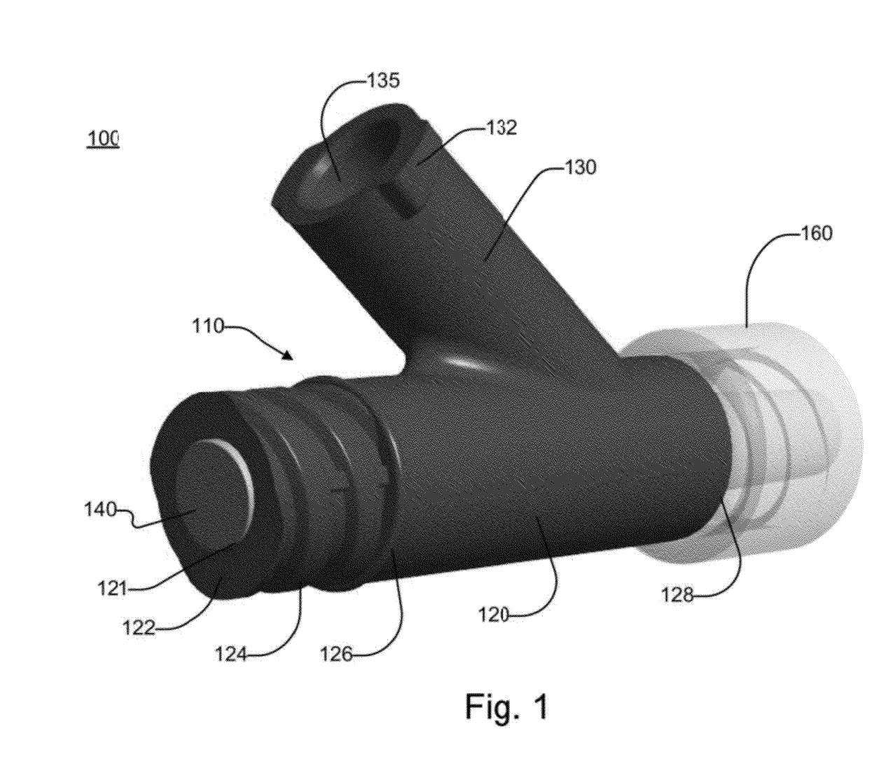

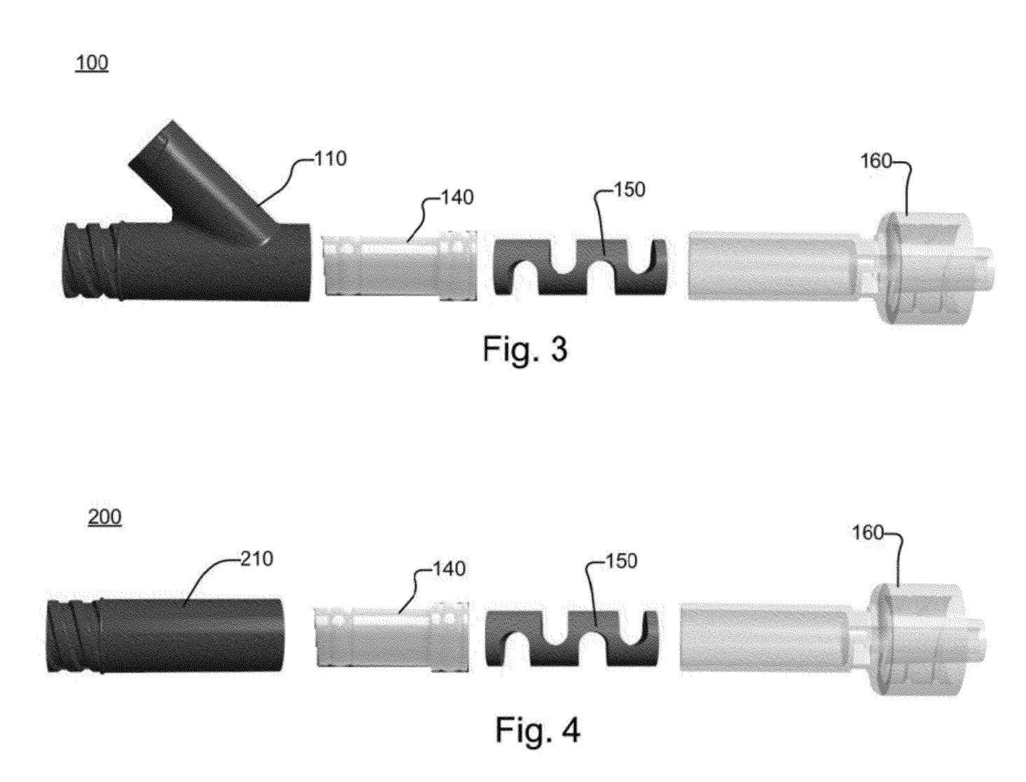

FIG. 1 shows a perspective view of an example of a valve system 100 that is constructed according to the principles of the disclosure. The valve system 100 includes a housing 110, a poppet 140, an elastoguide 150 (shown in FIG. 3), and an adaptor 160. The housing 110 may include a longitudinal housing portion 120 and an offset housing portion 130. The longitudinal housing portion 120 and the offset housing portion 130 may be configured in the shape of a Y, as seen in the figure.

The longitudinal housing portion 120 includes an opening 121 in a face 122 at one end to receive and allow a portion of the poppet 140 to pass through the opening 121. The longitudinal portion 120 includes another opening 128 at an opposite end to receive a portion of the adaptor 160. The longitudinal housing portion 120 may further include a fastener portion 124 that is configured to engage, for example, an intravenous supply line (not shown) that may include a female luer (not shown). The fastener portion 124 may include, for example, a threading (shown in FIG. 1), a groove, a tongue, a protrusion, or the like, which may engage and lock a corresponding fastener (not shown) that may be provided, for example, in a male luer (not shown). The male luer may include, for example, a slip and lock style luer. The housing portion 120 may further include a seal 126, which is configured to engage a part of the corresponding fastener (not shown) and provide an interference that prevents, for example, a male luer lock spin collar from prematurely unscrewing.

The offset housing portion 130 may include an opening 135 and a fastener 132. The fastener 132 may include, for example, a threading, a groove, a tongue, a protrusion (as shown in FIG. 1), or the like, which may engage and lock a corresponding fastener (not shown) that may be provided, for example, on an intravenous supply line (not shown), or a cap (not shown). The opening 135 may be used to bond or attach a tube segment as part of, for example, an IV set.

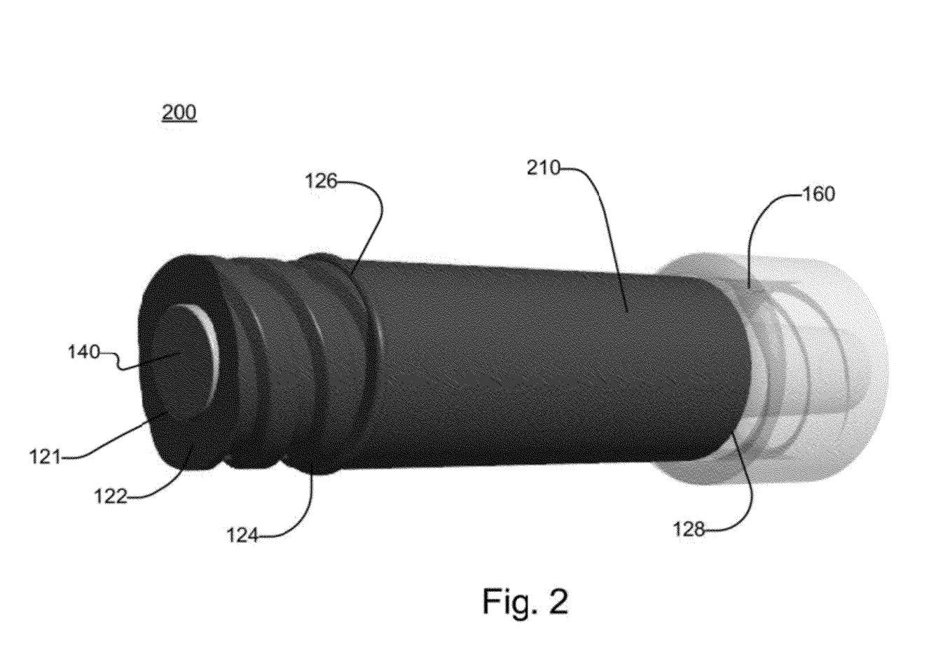

FIG. 2 shows a perspective view of another example of a valve system 200 that is constructed according to the principles of the disclosure. Further to description provided above with respect to FIG. 1, the valve system 200 includes a housing 210, the poppet 140, the elastoguide 150 (shown in FIG. 4), and the adaptor 160.

FIG. 3 shows an exploded view of the valve system 100, according to the principles of the disclosure.

FIG. 4 shows an exploded view of the valve system 200, according to the principles of the disclosure.

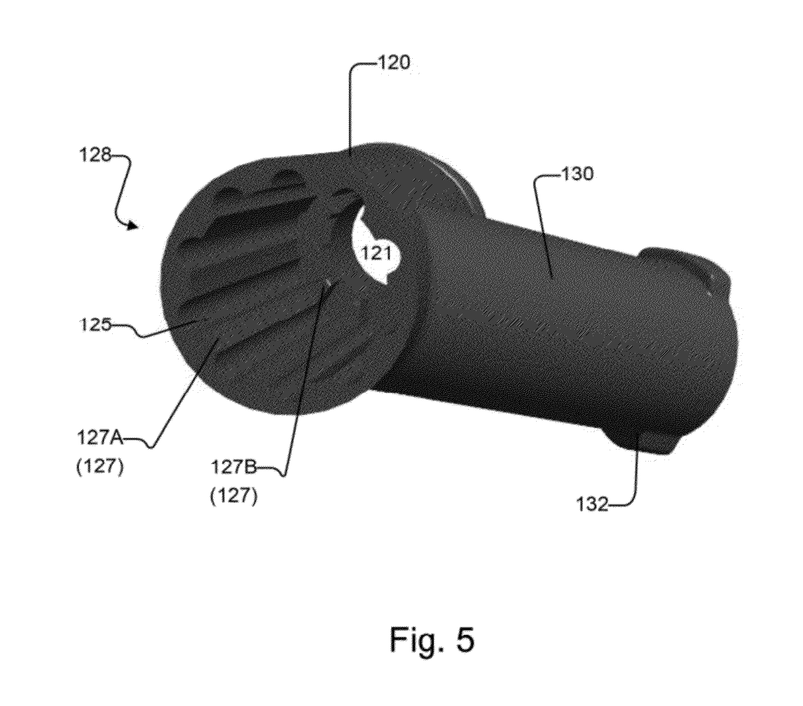

FIG. 5 shows another perspective view of the valve system 100. The housing portion 120 includes a plurality of channels 125 and a plurality of ribs 127. The channels 125 may each include a longitudinal recess that includes any configuration, including, but not limited to, for example, the semi-cylindrical shape shown in the figures, a triangular shaped-recess (not shown), rectangular shaped-recess, or the like, or any combination of the foregoing. The plurality of ribs 127 may each include a first guide portion 127A and a second guide portion 127B. Similarly, the ribs 127 may include any configuration that is capable of guiding and holding the adaptor 160 in the housing portion 120. The channels 125 are configured to provide a passageway between the housing portion 120 and the adaptor 160 for fluid to flow from the opening 121 to the opening 128, or visa-versa, in the housing portion 120. The ribs 127 are configured to guide and hold the adaptor 160 in the housing portion 120.

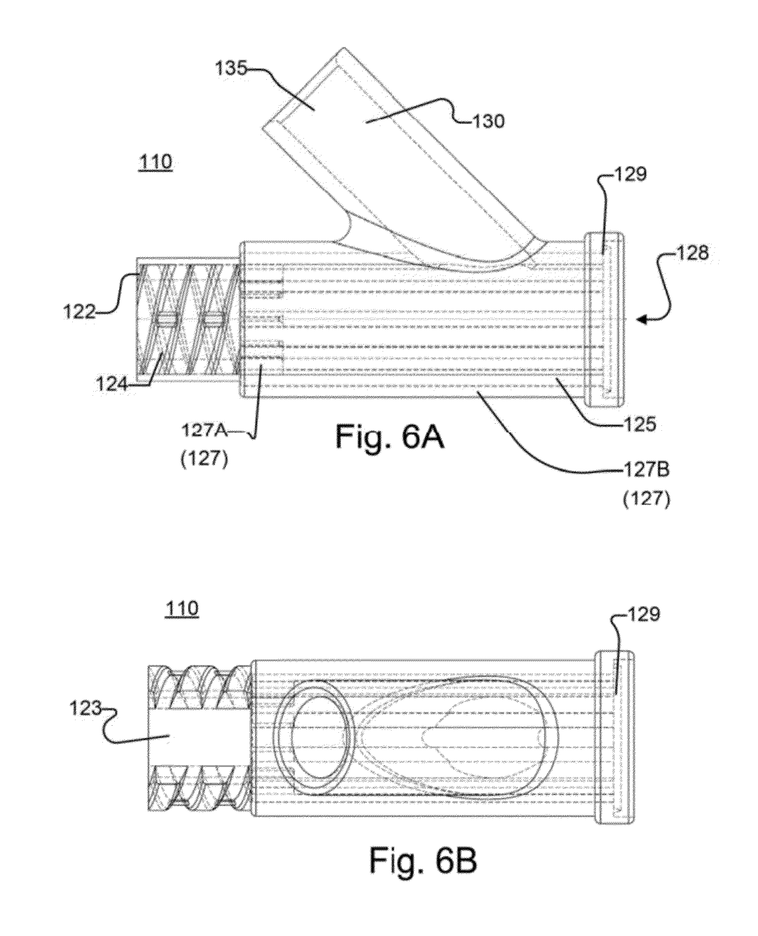

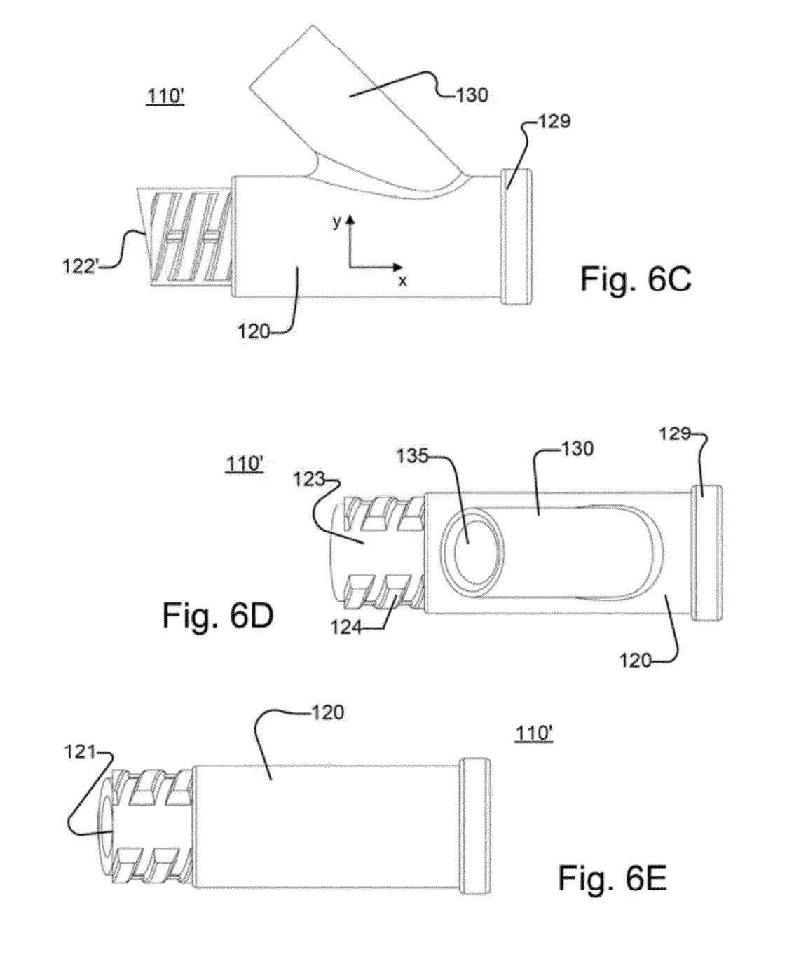

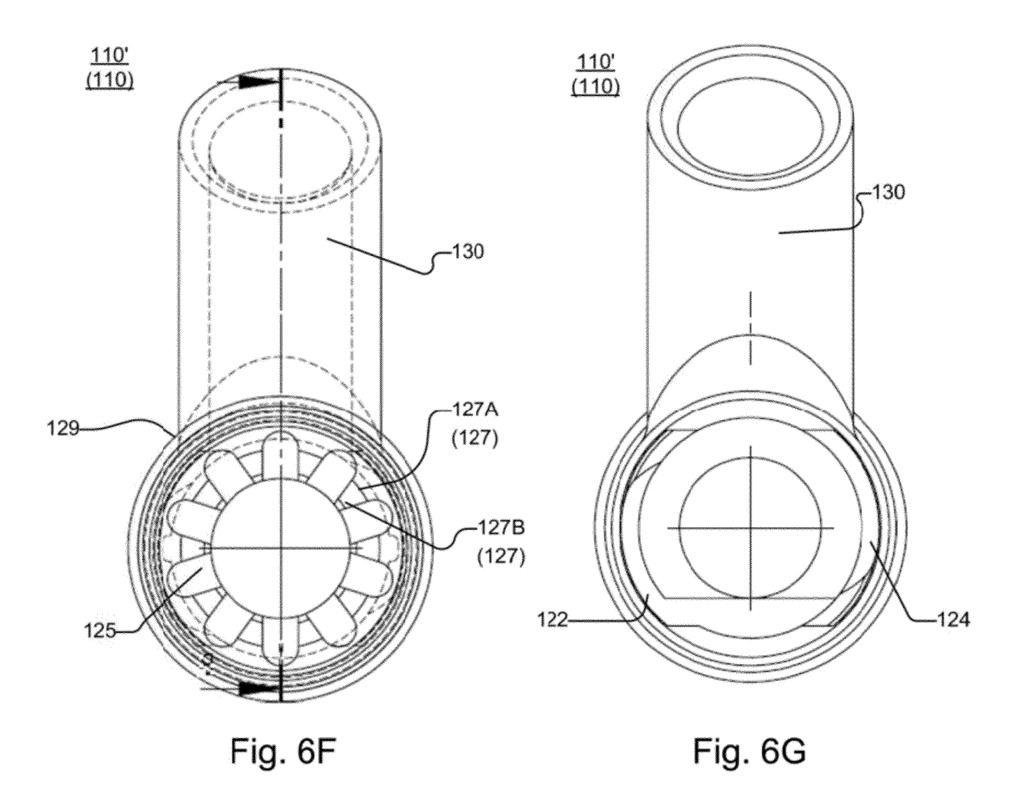

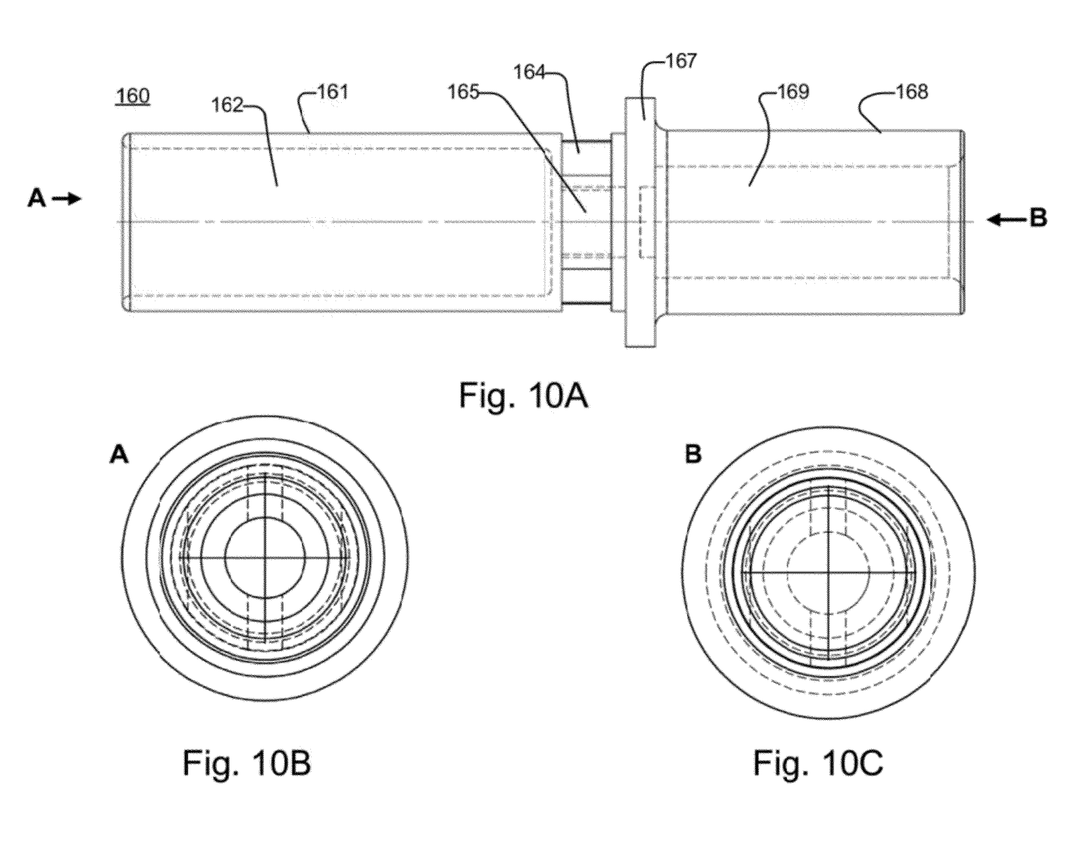

FIGS. 6A-6B show a side view and a top view, respectively, of the housing 110. As seen in FIGS. 6A-6B, the housing 110 may further include a fastener guide portion 123 and a collar 129. The fastener guide portion 123 may be configured to engage and properly align the corresponding fastener (not shown) that may be provided, for example, on the female luer (not shown). The collar 129 may be configured to sealably engage a flange portion 167 (shown in FIG. 10A) of the adaptor 160, so as to prevent any leakage of fluid from the valve system 100 (shown in FIGS. 1 and 3, or the valve system 200, shown in FIGS. 2 and 4). The housing 110 (or 210) may be integrally formed with the collar 129 by a process such as, for example, injection molding. Alternatively, the collar 129 may be provided as a seal-ring that may be attached to the housing 110 or the adaptor 160.

FIGS. 6C-6G show various views of a further example of a housing 110′ that is constructed according to the principles of the disclosure. In particular, FIG. 6C shows a side view of the housing 110′; FIG. 6D shows a top view of the housing 110′; and FIG. 6E shows a bottom view of the housing 110′. FIGS. 6F and 6G show a back view and a front view, respectively, of the housing 110′ (or 110). The housing 110′ differs from the housing 110 shown in FIGS. 6A and 6B in that the surface of the face 122′ is offset from the y-axis by a predetermined angle to substantially match the angle of the surface of the portion of the poppet 140 that passes through the opening 121. The offset angle may include an angle of, for example, between about 10° and about 15° from the y-axis.

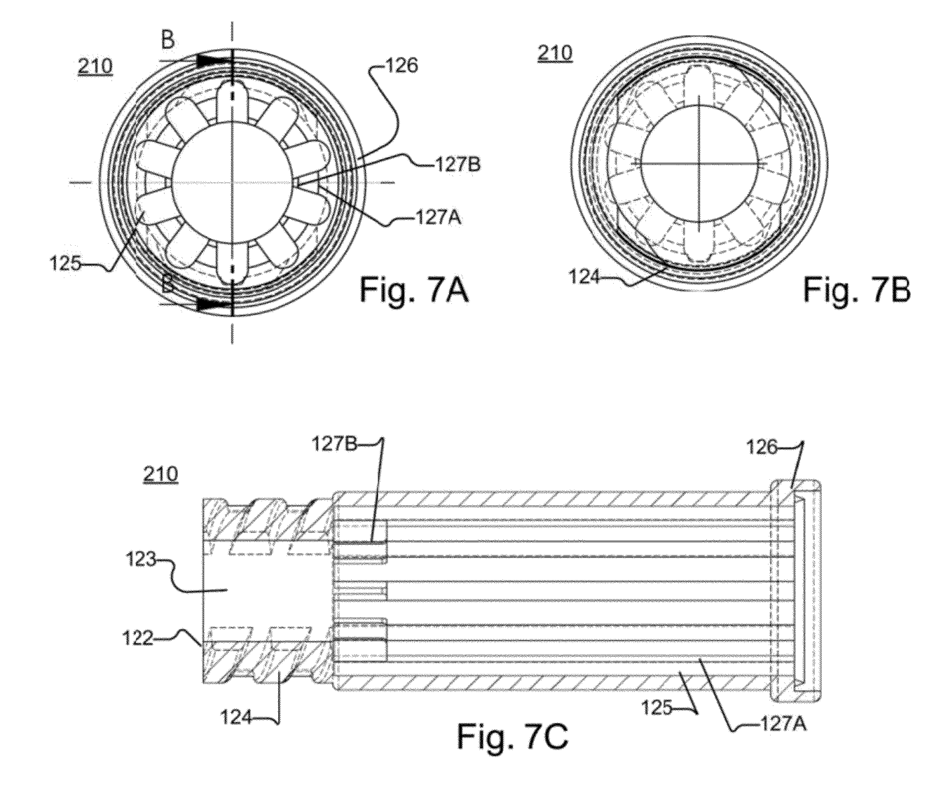

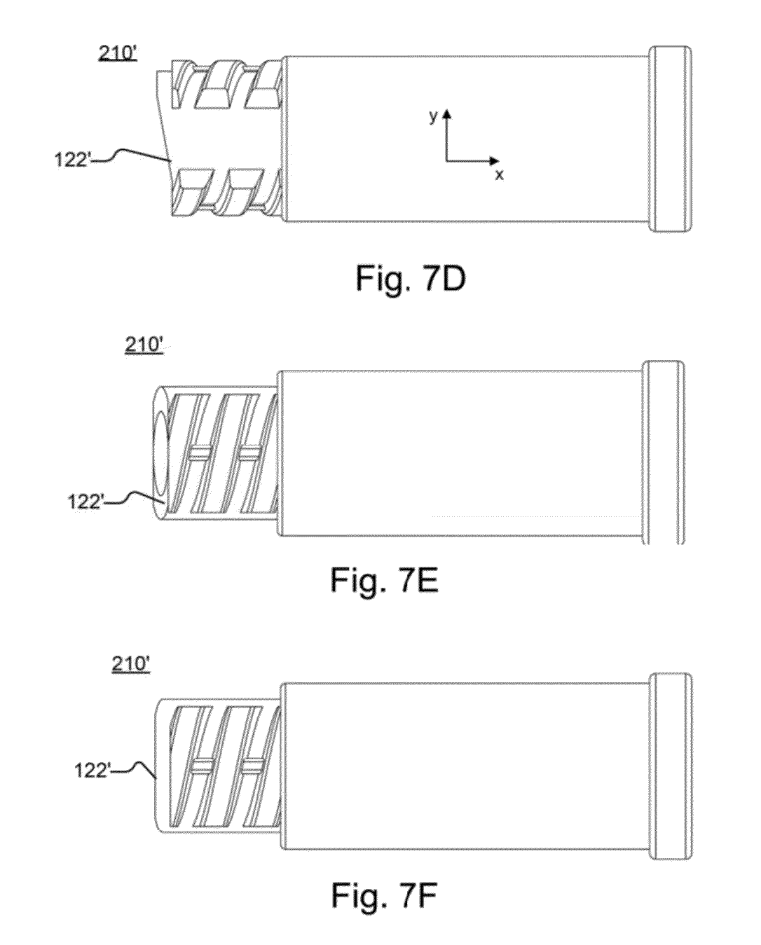

FIGS. 7A-7C show various views of the housing 210, according to principles of the disclosure. In particular, FIG. 7A shows a back view of the housing 210; FIG. 7B shows a front view of the housing 210; and FIG. 7C shows a side, cross-section view of the housing 210 cut along the plane B-B in FIG. 7A. As seen, the housing 210 may include the fastener guide portion 123 and the collar 129.

FIGS. 7D-7F show various views of another example of a housing 210′ that is constructed according to the principles of the disclosure. In particular, FIG. 7D shows a side view of the housing 210′; FIG. 7E shows a bottom view of the housing 210′; and FIG. 7F shows a top view of the housing 210′. The housing 210′ differs from the housing 210 shown in FIGS. 7A and 7C in that the surface of the face 122′ is offset from the y-axis by a predetermined angle to substantially match the angle of the surface of the portion of the poppet 140 that passes through the opening 121. The offset angle may include an angle of, for example, between about 10° and about 15° from the y-axis.

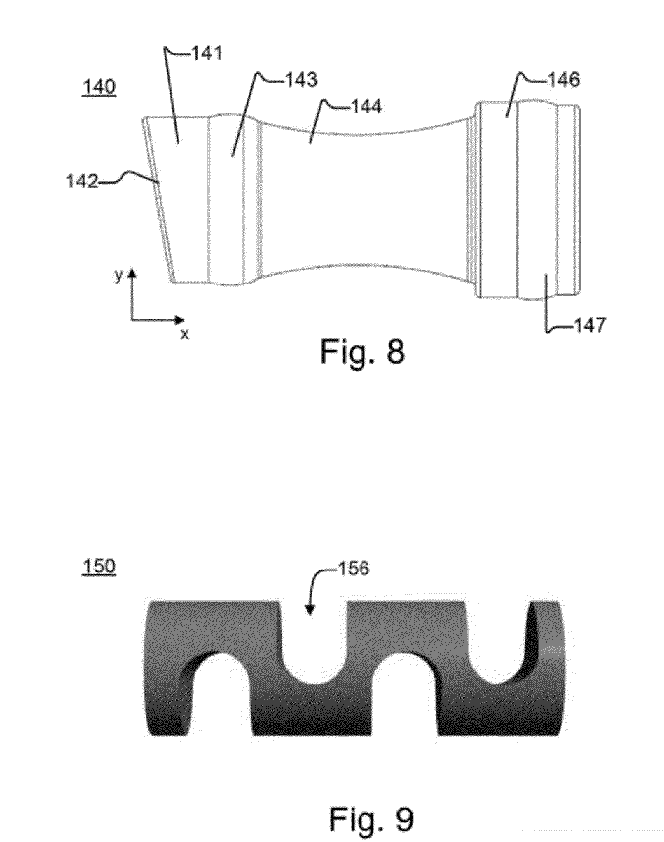

FIG. 8 shows an example of a poppet 140, which is constructed according to the principles of the disclosure. The poppet 140 may have a substantially hour-glass shape (as seen in FIG. 8), a cylindrical shape (not shown), or the like. The poppet 140 may include an angled face 142 at a housing-side end portion 141, such as, for example, an angle of between about 10° and about 15° from the y-axis. The angled face 142 may be configured to pass through the opening 121 in the housing 110 (110′, 210, or 210′). The poppet 140 may further include a sealing portion 143 that is configured to engage the inner walls of the housing 110 (110′, 210, or 210′) to provide a seal that prevents fluid from flowing through the opening 121 into the housing 110. The poppet 140 includes a middle portion 144 and an adaptor-side end portion 146. The middle portion 144 may have a reduced diameter, for example, to prevent the middle portion 144 from contacting the inner walls of the housing 120 (or 210) during axial loading, which may cause the middle portion 144 to bulge. The adaptor-side end portion 146 may include a sealing portion 147 that is configured to engage the inner walls of the adaptor 160 to provide a seal that prevents fluid from flowing into a cavity (not shown) that may be provided between the inner walls of the adaptor 160 and the end portion 146 of the poppet 140. The sealing portions 143, 147 may be integrally formed with the poppet 140, or the sealing portions 143, 147 may each include a seal that is affixed to the poppet 140. The end portion 146 may include a flat surface for contacting a surface of the elastoguide 150, or the end portion 146 may include an opening (not shown) for receiving a portion of the elastoguide 150.

FIG. 9 shows an example of an elastoguide 150, which is constructed according to the principles of the disclosure. The elastoguide 150 may be constructed from an elastic material, such as, for example, a silicone, or the like, that is capable of absorbing and storing compression energy and releasing the stored energy. The elastoguide 150 may include a plurality of recesses 154. The recesses 154 may have a semi-circular shape, a triangular shape, a square shape, or the like, or any combination of the foregoing. The elastoguide 150 may have an equal number of recesses 154 on each side (as shown in FIG. 9) to provide substantially balanced energy storage and release. Alternatively, the elastoguide 150 may be configured to include a plurality of annular portions (not shown) having different diameters, without departing from the scope or spirit of the disclosure.

Referring to FIGS. 8 and 9, the poppet 140 and the elastoguide 150 may be formed as a single structure using a process, such as, for example, injection molding. The poppet 140 portion and the elastoguide 150 may be molded from the same or different materials.

FIGS. 10A-10 show various views of an example of an adaptor 160, which is constructed according to the principles of the disclosure. In particular, FIG. 10A shows a side view of the adaptor 160; FIG. 10B shows a housing-side A view of the adaptor 160; and FIG. 10C shows a coupling-side B view of the adaptor 160. The adaptor 160 includes a housing-side portion 161, a mid-portion 164 and a coupling-side portion 168. The housing-side portion 161 includes a cavity 162 that is configured to receive and hold the elastoguide 150 and a portion of the poppet 140, which may be inserted partially in the cavity 162. The mid-portion 164 may include one or more openings 165 and a flange 167. The opening(s) 165 are configured to allow fluid to flow from the channels 125 into a channel 169 provided in the coupling-side portion 168. The flange 167 may be configured to serve as a stop and to provide a seal between the housing 110 (110′, 210, or 210′) to prevent any fluid from leaking. The coupling-side portion 168 may be coupled directly to, for example, a catheter, or, as seen in FIG. 1, a female coupler may be affixed to the coupling-side portion 168, to allow for coupling to a male luer (not shown).

Referring to FIG. 10A, the inner walls of the cavity 162 may be configured so that the diameter of the cavity 162 gradually diminishes from the opening of the cavity 162 to the mid-portion 164 of the adaptor. This semi-conical shape (not shown) may provide for easier removal of the adaptor 160 from a mold during manufacturing. This shape may also provide reduced drag on the poppet 140 during operation of the of the valve system 100, when the poppet 140 moves from an open configuration to a closed configuration (shown in FIG. 1), since the poppet 140 will experience a larger diameter as it moves to the closed configuration (or position), at the same time at which the elastoguide 150 will provide a reduced pushing force on the poppet 140.

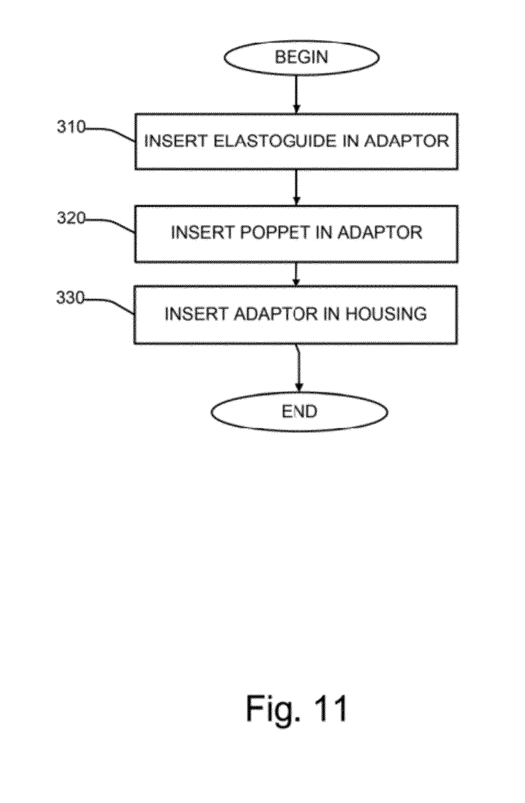

FIG. 11 shows a process for assembling the valve system of FIG. 1 or FIG. 2. Referring to FIGS. 1-3, 5, 10A and 11 concurrently, the valve system 100 (or 200) may be assembled by, for example, inserting the elastoguide 150 into the adaptor 160 (Step 310). The poppet 140 may then be inserted into the cavity 162 in the housing-side portion 161 of the adaptor 160 (Step 320). The housing-side portion 161, including the poppet 140 and the elastoguide 150, may be inserted into the housing 110 (110′, 210, or 210′) (Step 330). The housing 110 (110′, 210, or 210′) may be configured to engage and lock to the adaptor 160, so as prevent unintentional separation of the adaptor 160 from the housing 110.

Once assembled, the valve system 100 (or 200) may function to allow fluid to flow into the opening 121 when the poppet 140 is engaged and forced sufficiently into the housing 110 (110′, 210, or 210′). The fluid may then flow through the opening 121 and into one or more of the channels 125 of the housing 110. The fluid may flow in the channels 125, between the inner walls of the housing 110 (110′, 210, or 210′) and the external walls of the housing-side portion 161 of the adaptor 160, to the openings 165 in the mid-portion 164 of the adaptor 160. The fluid may then flow from the opening 165 into the channel 169 and out from the adaptor 160.

The embodiments of the valve system 100 (or 200) described herein are resistant to cracking, especially since lubricants, such as, for example, silicone oil, are not necessary for operation. The cross-section of the leer connection (for example, the fastener portion 124) may be maximized for added strength. The configuration of the valve system 100 (or 200) maximizes flow volume while minimizing the amount of material used to construction the valve system 100 (or 200).

Since the valve system 100 (or 200) may be made entirely from plastic or other synthetic materials, including, for example, non-metallic materials, the valve system 100 (or 200) may be used in MRI/NMR applications.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications or modifications of the disclosure.

What is claimed is:

1. A valve system, comprising:
a housing that includes an opening for receiving or ejecting a fluid;
a poppet that is configured to control a flow of the fluid through the opening;
an elastoguide that is configured to drive the poppet to a closed position; and
an adaptor that is configured to receive and hold the poppet and the elastoguide.

2. The system according to claim 1, wherein the housing comprises:
a longitudinal housing portion and an offset housing portion, the longitudinal housing portion comprising an angle of between about 10° and about 15°.

3. The system according to claim 1, wherein the housing comprises:
a fastener portion that is configured to engage and lock to a male luer.

4. The system according to claim 1, wherein the housing comprises:
a plurality of channels that are configured to guide the fluid from one end of the housing to an opposite end of the housing.

5. The system according to claim 1, wherein the housing comprises:
a plurality of ribs that are configured to guide and hold the adaptor in the housing and to provide a positive stop for the poppet in the closed position.

6. The system according to claim 1, wherein the poppet comprises:
a substantially hour-glass shape.

7. The system according to claim 1, wherein the poppet comprises:
an angled face at a housing-side end portion.

8. The system according to claim 3, wherein the male luer comprises:
a slip and lock style luer.

9. The system according to claim 1, wherein the poppet comprises:
a surface that is configured to engage a portion of the elastoguide.

10. The system according to claim 1, wherein the elastoguide comprises:
an elasic material.

11. The system according to claim 1, wherein the elastoguide comprises:
a recess that is configured reduce overall weight of the elastoguide or to provide additional compressibility.

12. The system according to claim 1, wherein the elastoguide comprises:
a substantially longitudinal shape.

13. The system according to claim 1, wherein the adaptor comprises:
a cavity that is configured to receive and hold a portion of the poppet.

14. The system according to claim 13, wherein the cavity is further configured to receive and hold the elastoguide.

15. The system according to claim 1, wherein the adaptor comprises:
an opening that is configured to receive the fluid.

16. The system according to claim 15, wherein the opening is in fluid communication with a channel in the housing and a channel that is provided in the adaptor.

17. The system according to claim 1, wherein the adaptor comprises:
a flange that engages a portion of the housing.

18. A valve system, comprising:
a housing that includes an opening for receiving or ejecting a fluid;
a poppet that is configured to control a flow of the fluid through the opening; and
an elastoguide that is configured to drive the poppet to a closed position,
wherein the elastoguide includes a recess that is configured to reduce overall weight of the elastoguide or to provide additional compressibility.

19. The system according to claim 18, further comprising:
an adaptor that is configured to hold the elastoguide.

20. A method for assembling a valve system, the method comprising:
inserting an elastoguide in an adaptor;
inserting the poppet in the adaptor with the elastoguide; and
inserting the adaptor with the poppet and the elastoguide into a housing.
